(19) United States
(12) Patent Application Publication
(10) Pub. No.: US 2018/0168915 A1
(43) Pub. Date: Jun. 21, 2018

(54) CUPPING APPARATUS

(71) Applicant: Patrick GWEN, Houston, TX (US)

(72) Inventor: Patrick GWEN, Houston, TX (US)

(21) Appl. No.: 15/385,734

(22) Filed: Dec. 20, 2016

(51) Int. Cl. A61H 9/00; A61M 1/08

(52) U.S. Cl. CPC A61H 9/0057; A61H 2201/1409; A61M 1/08

(57) ABSTRACT

The apparatus for cupping includes a cupping bell, a suction chamber, and a piston member with a handle and piston skirt. The suction chamber connects to the cupping bell, and actuation of the piston member through the suction chamber creates a vacuum in the cupping bell. Skin and soft tissue adjacent to the opened end of the cupping bell is suctioned into the cupping bell for cupping therapy of the treatment area. The suction chamber has a locking device cooperative with a locking notch on the piston so that dimensions of the piston member correspond to set levels of suction in the cupping bell with continuous pressure when increasing suction during a treatment. The cupping bell has a flared portion for engaging the treatment area with less trauma and pain for the patient.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to an apparatus for use in cupping treatments or cupping therapy. In particular, the present invention relates to an apparatus with a suction chamber separate from a cupping bell portion on the skin.

### 2. Description of Related Art Including Information Disclosed Under 37 CFR 1.97 and 37 CFR 1.98.

Cupping treatment or cupping therapy involves localized suction on an area of soft tissue. The blood vessels, lymphatic vessels, connective tissue, and muscles are stimulated by the negative pressure to increase circulation of blood and lymph. The vacuum expands capillaries and small blood vessels to improve flow at the surface of the skin. Cupping therapy or “cupping” is an alternative form of medicine with an extensive history dating back several centuries.

Traditional methods rely on heated air inside a cup or ball, which is then placed against the skin. As the air cools inside the cup, the skin is pulled into the cup to reduce volume of the air, such that a vacuum is formed in the interior volume of the cup and the skin. More recent technology dispenses with the use of heat, especially the traditional open flame. In particular, a mechanical suction pump can be used to create the vacuum.

U.S. Pat. No. 9,095,647, issued to Lee, et al. on 4 Aug. 2015, discloses a disposable cupping apparatus with an inner diaphragm to create the suction. The actuation of the inner diaphragm mechanically creates the vacuum, and the resilient cupping bell holds the vacuum until the end of the treatment.

U.S. Pat. No. 5,662,677, issued to Wimmer on 2 Sep. 1997, describes a cupping instrument with a cylindrical tubular extension extending from the cupping bell. A piston in the tubular extension mechanically creates the vacuum, and a valve between the tubular extension and the cupping bell closes to set the amount of vacuum in the cupping bell. The piston can be actuated by a pulling action or rotating action along threads on the length of the piston. The valve is manually operated by a one way trigger.

There are problems with the mechanical creation of the vacuum. The actuation of the piston lacks control. In threaded pistons, the piston must be rotated along threads to decrease the volume and to create the vacuum. The threaded piston can be locked, but the actuation is dependent on the dimension of the threads. The rate of applying the amount of suction and releasing the amount of suction depends on the size of the threads. Releasing the vacuum requires unscrewing the piston, such that the relief to the skin can be very slow and gradual instead of immediate as required in cupping treatments. Also, a painful application cannot be released efficiently. In valve systems, such as U.S. Pat. No. 5,662,677, the valve can be triggered at any point of actuation of the piston. There is a lack of control. A strong pull on the piston may overshoot the desired level of suction for a painful experience. There is a lack of consistency, such that the cupping treatment with different suction in each cupping bell is difficult to record and track for an individual patient. Whether a patient received a beginner level of vacuum or an advanced level of vacuum is difficult to track. Furthermore, the one-way valve prevents re-setting the vacuum. For example, an initial vacuum cannot be increased to another treatment level without releasing the entire initial vacuum. Once the valve is closed, the vacuum is released by depressing on the skin adjacent the cupping bell or releasing valve. However, there are treatments that require starting at an initial vacuum and progressing to a higher level of suction during the treatment without releasing the initial vacuum. Also, the trauma to the skin adjacent to the cupping bell is a continuing problem of cupping therapy, especially when mechanically induced vacuum depend on this trauma to release the vacuum.

It is an object of the present invention to provide an embodiment of an apparatus for cupping with mechanically created vacuum.

It is another object of the present invention to provide an embodiment of an apparatus for cupping with a suction chamber with a piston member to create a vacuum in a cupping bell.

It is still another object of the present invention to provide an embodiment of an apparatus for cupping with a locking means on a suction chamber to create pre-determined levels of suction in a cupping bell cooperative with the piston member.

It is another object of the present invention to provide an embodiment of an apparatus for cupping with a suction chamber with a flared portion to create a vacuum in a cupping bell.

It is still another object of the present invention to provide an embodiment of an apparatus for cupping with a flared portion on the suction chamber to control skin displacement in the cupping bell.

These and other objectives and advantages of the present invention will become apparent from a reading of the attached specification.

### BRIEF SUMMARY OF THE INVENTION

Embodiments of the apparatus for cupping create a vacuum by mechanical action of a piston. Instead of relying upon volatile open flames and heat, negative pressure can be created by displacement of a piston within a cylinder. Additionally, the amount of suction can be consistently varied by pre-determined amounts of displacement of the piston. The apparatus for cupping can include a cupping bell, a suction chamber, and a piston member. The suction chamber is in fluid connection with the closed end of the cupping bell and has a locking means cooperative with the piston member. In particular, the locking means is cooperative with a locking notch of the piston member. The piston member housed in the suction chamber is movable between the bottom end and the top end of the suction chamber. The piston member can further include a piston skirt in sealing engagement to the suction chamber, and a handle extending out of the top end of the suction chamber.

In some embodiments, the piston member includes a shaft and a first plurality of ribs radially mounted around the shaft. In a first configuration of the piston member, the locking means aligns with the locking notch so that the shaft and ribs pass through the locking means when creating a first vacuum. In a second configuration of the piston member, the ribs no longer fit through the locking means. The displacement of the piston member is set to a first vacuum in the cupping bell. In further embodiments, there is a second plurality of ribs, so that the second plurality of ribs can pass through the slot for another displacement corresponding to a second vacuum with greater suction than the first vacuum. The ribs of the second plurality no longer fit through the locking means for the second vacuum. The treatment area maintains a vacuum between configurations, and the length dimensions of the ribs consistently set the vacuums and difference between the first and second vacuum. The locking means can be a slot corresponding to a cross-section of the shaft and the first plurality of ribs, a switch with insert to extend into the locking notch from the suction chamber, or other known mechanical lock.

Variations include the apparatus including a spring between the locking plate and the piston skirt. The spring can be actuated to assist in separating the treatment area from the cupping bell. There can also be a flared portion on the opened end of the cupping bell. There are variations of the flared portion with a different sizes and proportions to the cupping bell. The flared portion stabilizes the skin of the treatment area for less pain with gradual tightening of the treatment area at the cupping bell.

Embodiments of the present invention include a method for cupping with the apparatus. The cupping bell is set against a treatment area of a patient, and the piston member actuates from the bottom end toward the top end of the suction chamber. The locking means engages the locking notch of the piston member to create an initial vacuum with the treatment area and the cupping bell. The initial vacuum is consistent because the displacement of the piston member is set by the dimensions of the piston member, such as the length of ribs when the piston member is comprised of a shaft and a first plurality of ribs radially mounted around the shaft. There is a first configuration with the locking means aligned with the locking notch so as to pass the shaft through the locking means when creating a first vacuum, which can also be the initial vacuum. There can also be a second configuration with the locking means engaged to the locking notch so as to hold the first plurality of ribs against the locking means after passing through the locking means. The length of the ribs determines displacement of the piston and the amount of vacuum in the cupping bell. Alternating pulling and engaging (by twisting or actuating the switch) the piston member can control the increase in suction and holding the vacuum stable with more than one plurality of ribs.

The method further includes embodiments with maintaining a continuous vacuum when the piston member moves from the second configuration to the fourth configuration. Still other embodiments include releasing the vacuum by actuating a spring for popping the cupping bell from the treatment area.

### BRIEF DESCRIPTION OF THE SEVERAL VIEWS OF THE DRAWINGS

FIG. 1 is a front elevation view of an embodiment of the apparatus for cupping according to the present invention.

FIG. 2 is an upper perspective view of an embodiment of the apparatus for cupping according to the present invention.

FIG. 3 is a cross-section view along a longitudinal axis of the piston member, showing an embodiment of the apparatus with a spring.

FIG. 4 is a cross-section view along a longitudinal axis of the piston member, showing a first configuration with the shaft passing through the locking means and another embodiment of the flared portion.

FIG. 5 is a cross-section view along a longitudinal axis of the piston member, showing a second configuration with the first plurality of ribs locked relative to the locking means.

FIG. 6 is a cross-section view along a longitudinal axis of the piston member, showing a third configuration with the second plurality of ribs passing through the locking means.

FIG. 7 is a cross-section view along a longitudinal axis of the piston member, showing a fourth configuration with the second plurality of ribs held relative to the locking means, consistent with FIG. 2 and still another embodiment of the flared portion.

FIG. 8 is a cross-section view along a longitudinal axis of the piston member, showing a second configuration with the first plurality of ribs locked relative to another embodiment of a locking means.

### DETAILED DESCRIPTION OF THE INVENTION

Cupping therapy previously relied upon expertise and dexterity of the individual therapist to control the amount of suction of each cup. The skill in managing ignition by open flame to heat air within each cup required practice and experience. As a result, mechanically creating the suction developed to increase accessibility of safe cupping therapy. The known mechanical cupping systems have relied on threaded engagement and pulling action, but lack control and consistency. Just as heating to the correct amount for the correct vacuum required expertise, mechanically creating a consistent vacuum required expertise.

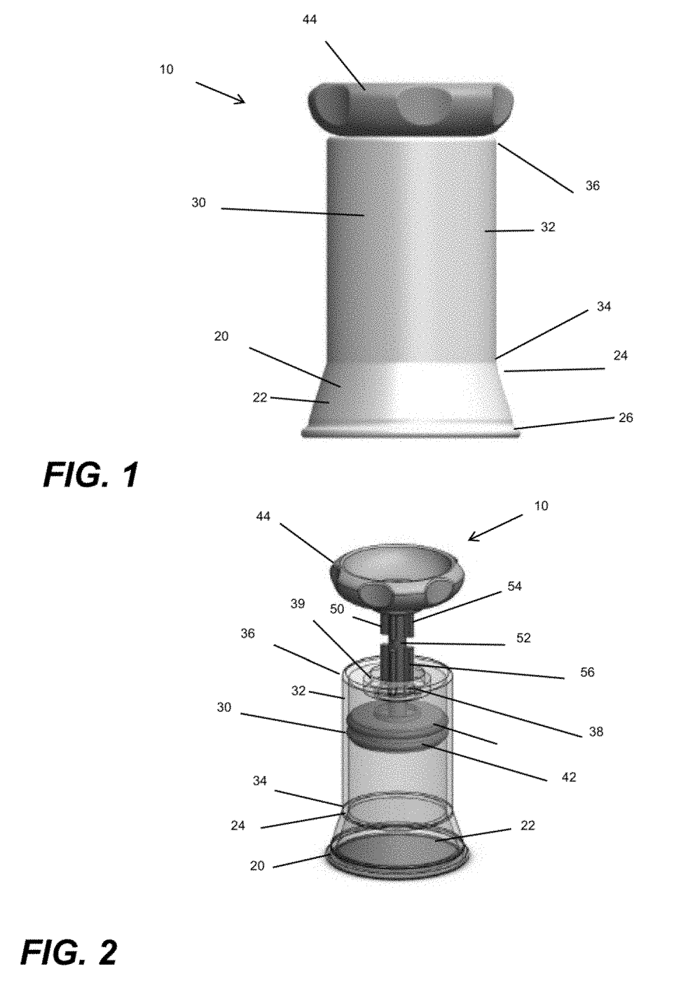

Referring to FIGS. 1-8, the present invention includes embodiments of an apparatus 10 for cupping. The apparatus 10 allows for consistent pre-determined vacuum levels to be created by mechanical action. Less training is required, and a patient may be able to operate the apparatus 10 on himself or herself, depending upon the location of the treatment area on the body. The apparatus 10 includes a cupping bell 20, a suction chamber 30, and a piston member 40. The cupping bell 20 has a generally cylindrical body 22, and there is a closed end 24 and an opened end 26. The opened end 26 faces the treatment area on the body of the patient. The suction chamber 30 is in fluid connection with the closed end 24 so that a vacuum created by in the suction chamber corresponds to a vacuum in the cupping bell 20. The suction chamber 30 has a generally tubular body 32 with a bottom end 34 and a top end 36. In some embodiment, the bottom end 34 can extend below the closed end 24 and into the volume of the cupping bell 20. FIGS. 1-7 also show one embodiment of a locking means 38 of the suction chamber 30 between the bottom end 34 and the top end 36. FIG. 8 shows another embodiment of another locking means 38′. The piston member 40 is housed in the suction chamber 30 and movable back and forth between the bottom end 34 and the top end 36. There is a locking notch 50 on the piston member 40 between the handle 44 and the piston skirt 42. The locking means 38, 38′ of the suction chamber 30 is cooperative with the locking notch 50 of the piston member 40.

FIGS. 1-8 show an embodiment of the piston member 40 comprising a piston skirt 42, a handle 44, and a locking notch 50 between the piston skirt 42 and the handle 44. The piston skirt 42 is in sealing engagement to walls of the suction chamber 30. Suction is created by increasing the volume for the air in the cupping bell 20. Embodiments of the piston skirt 42 include at least one 0-ring for the seal against the tubular body 32 of the suction chamber 30. FIGS. 1-8 show two 0-rings around an end of the piston member 40 at the bottom end 34. The handle 44 is shown extending out of the top end 36 of the suction chamber 30. The handle 44 can be gripped by the hand of the user, and the shape and dimensions facilitate grasping for pulling and rotating the piston member 40. FIGS. 1-2 show a polygonal shape with sides for grasping by hand.

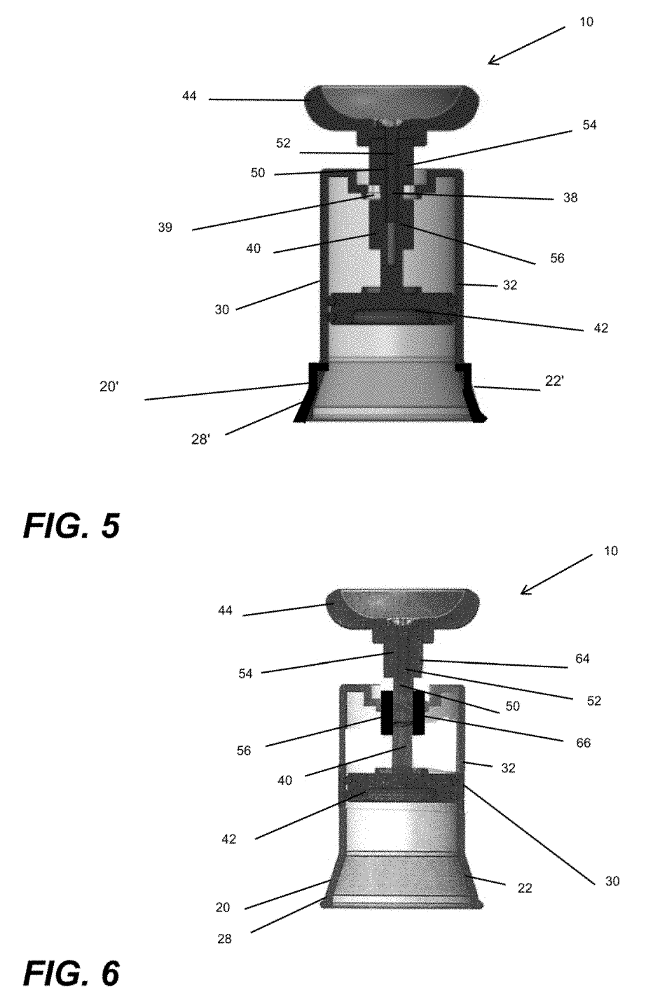

Embodiments of the locking notch 50 include a mechanical interaction for the position of the piston member 40 relative to the bottom end 34 of the suction chamber 30. In one embodiment, the piston member 40 includes the locking notch 50 between the piston skirt 42 and the handle, a shaft 52 and a first plurality 54 of ribs radially mounted around the shaft 52. The first plurality 54 of ribs is between the locking notch 50 and the handle 44. FIG. 4 shows the piston member 40 in a first configuration with the locking means 38 aligned with the locking notch 50 so as to pass the shaft 52 through the locking means 38. Pulling action on the piston member 40 creates a first vacuum in the cupping bell 20. FIG. 5 shows the piston member 40 in a second configuration with the locking means 38 engaged to the locking notch 50 to hold the first plurality 54 of ribs relative to the locking means 50. FIG. 5 shows the ribs friction fit against the locking means 38. The pulling action for the first vacuum set in the cupping bell 20 is now controlled. The pulling action is no longer variable. The amount of pulling action is determined by the size of the first plurality 54 of ribs. The first vacuum with a set amount of suction is standardized.

FIGS. 2 and 4-5 show an embodiment of the locking means 38 comprised of a slot 39 corresponding to a cross-section 56 of the shaft and the first plurality of ribs. The first configuration has the shaft 52 and first plurality 54 of ribs aligned with the slot 39 (FIG. 4). The second configuration has the first plurality 54 of ribs in friction fit engagement to the slot 39 by rotation of the shaft 52 (FIG. 5). Action on the piston member 40, after the initial pulling action, holds the first plurality 54 of ribs against the locking means 38 after passing through the slot 39. The ribs are friction fit against the locking means 38 because the slot 39 and ribs are no longer aligned.

FIG. 8 shows another embodiment of the locking means 38′ comprised of a switch 39′ and an insert 39″. The first configuration has insert 39″ in the retracted position so that the shaft 52 and first plurality 54 of ribs align with the locking means 38′. The second configuration has the first plurality 54 of ribs in friction fit engagement to the insert 39″ in the extended position by actuation of the switch 39′ (FIG. 8). Triggering the switch 39′ to actuate the insert 39″ relative to the piston member 40, after the initial pulling action, holds the first plurality 54 of ribs against the locking means 38′ after passing through the locking means 38′. The ribs are still friction fit against the locking plate 38 because insert 39″ blocks the ribs. Other known mechanical locks can be the locking means 38, 38′ of the present invention, including spring loaded triggers, lever, and other switches to hold the piston member 40 in placed relative to the suction chamber 30.

FIGS. 2-8 show embodiments of each of the ribs of the first plurality 54 of ribs as a flange 64. Each flange 64 of the first plurality 54 of ribs has set dimensions, including a first length extending along the shaft 52. The first length allows for mechanical control of the pulling action. The first vacuum is consistently determined by a displacement of the piston member 40 according to the first length. In this manner, each apparatus 10 can be uniformly and consistently applied to treatment areas on the body.

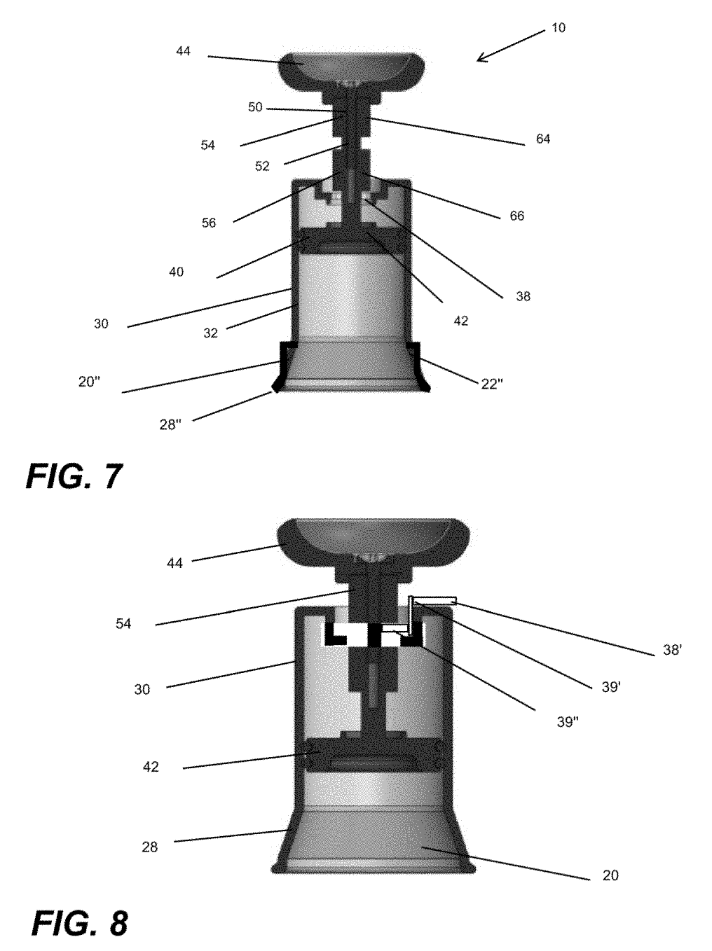

Another embodiment of the piston member 40 includes a second plurality 56 of ribs radially mounted around the shaft 52, as shown in FIGS. 2 and 4-7. The second plurality 56 of ribs is positioned between the first plurality 54 of ribs and the piston skirt 42. The third configuration has the shaft 52 and second plurality 56 of ribs aligned with the slot 39 (FIG. 6). The fourth configuration has the second plurality 56 of ribs in friction fit engagement to the slot 39 by rotation of the shaft 52 (FIG. 7). Action on the piston member 40, after another pulling action, holds the second plurality 54 of ribs against the locking means 38 after passing through the slot 39. The ribs are friction fit against the locking means 38 because the slot 39 and ribs are no longer aligned.

When the embodiment of the locking means 38 is the slot 39 of FIGS. 4-7, another twisting action and another pulling action pass the shaft 52 and second plurality 56 of ribs through the slot 39 so as to create a second vacuum in the cupping bell 20. FIGS. 2 and 7 show the piston member 40 in a fourth configuration with the locking notch 50 misaligned with the slot 39. In this embodiment, an additional twisting action on the piston member 40, after the other twisting action and the other pulling action, holds the second plurality 56 of ribs against the locking means 38 after passing through the slot 39. The ribs are friction fit against the locking means 38 because slot 39 and ribs are no longer aligned. The additional pulling action for the second vacuum set in the cupping bell 20 is also controlled. The additional pulling action is no longer variable, similar to the pulling action to set the first vacuum. The amount of the additional pulling action is determined by the size of the second plurality 56 of ribs. The second vacuum with a set amount of suction is also standardized. The suction of the second vacuum is greater than the suction of the first vacuum because the amount of volume created for the air in the cupping bell 2 is increased. In the embodiments of FIGS. 4-7, the slot 39 also corresponds to a cross-section of the shaft 52 and the second plurality 56 of ribs. In the embodiment of FIG. 8, the insert 39″ would be able to engage the second plurality 56 of ribs for a friction fit hold.

FIGS. 2-8 show embodiments of each of the ribs of the second plurality 56 of ribs as a flange 66. Each flange 66 of the second plurality 54 of ribs has set dimensions, including a second length extending along the shaft 52. The second length also allows for mechanical control of the additional pulling action. The second vacuum is consistently determined by a displacement of the piston member 40 according to the second length. The first length of each flange 64 of the first plurality 54 of ribs determines the first vacuum, and the second length of each flange 66 of the second plurality 56 of ribs determines the second vacuum.

FIGS. 2 and 4-7 show an embodiment of the locking means 38 with the cross-section of the shaft and the first plurality of ribs identical to the cross-section of the shaft and the second plurality of ribs, such that the second configuration corresponds to the fourth configuration. Similar twisting action controls the transition between the first configuration to the second configuration and the transition between the third configuration to the fourth configuration. When identical, the same amount of rotation of the shaft 52 can be used to lock and unlock the piston member 40 between configurations. However, other relationships between cross-sections are possible within the scope of the invention. The cross-section of the shaft and the first plurality of ribs can be different than the cross-section of the shaft and the second plurality of ribs. Different twisting action can differentiate between the configurations of the piston member 40 relative to the cupping bell 20. In the embodiment with each rib being a flange 64, 66, each flange 64 of the first plurality 54 of ribs is evenly distributed around the shaft 52, and each flange 66 of the second plurality 56 of ribs is also evenly distributed around the shaft 52. When evenly distributed, the direction of rotation can be either clockwise or counterclockwise. There can also be different patterns so that the twisting action may need to be in a particular direction to correspond to the proper cross-section and slot for the pre-determined vacuum and level of suction.

Additional pluralities of ribs can be set on the shaft 52 in other embodiments. The apparatus can have a third vacuum, fourth vacuum, etc., according to the number of pluralities. An apparatus can be specialized from beginner to advanced levels of suction. Additionally, the dimensions of the ribs can be different, such as different lengths of flanges. The increase in suction from the first vacuum to the second vacuum can be adjusted for beginner and advanced increases in levels of suction. There can be incremental increases as the treatment is suited to the user.

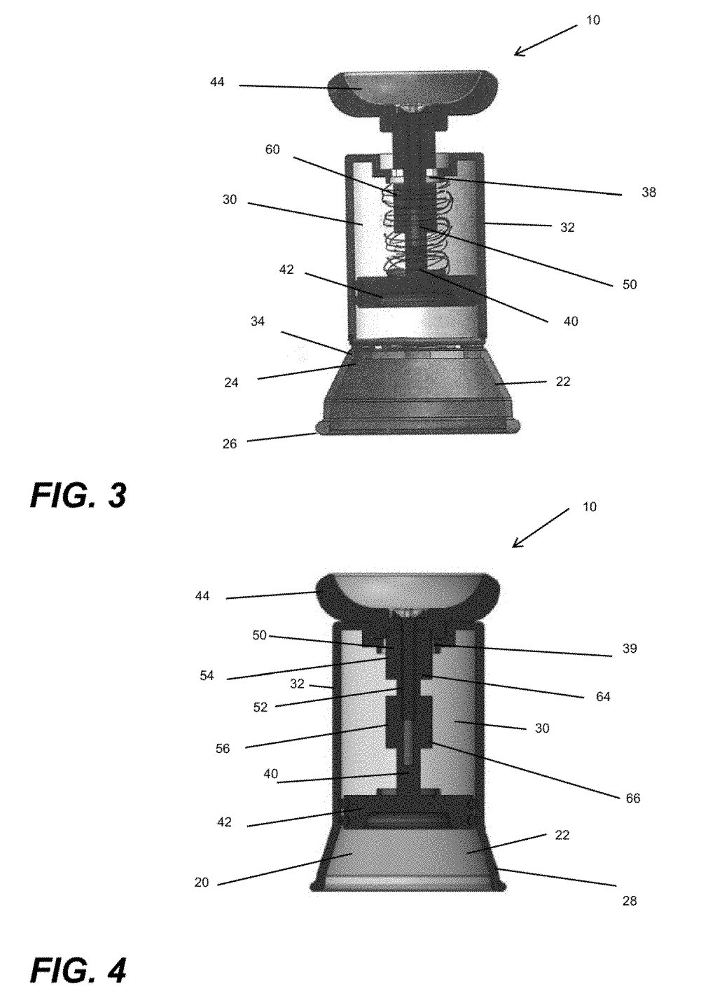

FIG. 3 shows an alternative embodiment with a spring means 60 between the locking means 38 and the piston skirt 42. When releasing either the first vacuum or the second vacuum, the spring means 60 can exert a force against the piston skirt 42. The additional force can trigger the release of the treatment area from the cupping bell 20. The force from the spring means 60 releases pressure by reducing the volume for the air of the cupping bell 20. The cupping bell 20 no longer holds the treatment area as tightly and can release each other. The release can be quicker than the existing threaded pistons, which depend on the size of threads and speed of rotation around the threads. The spring means 60 can be a coiled spring or other known tensioned element with stored potential energy. FIG. 3 shows a coiled spring extended in the first configuration and compressed in the second configuration. To unlock from the second configuration, the piston member 20 is twisted to align with the slot 39, and the first plurality 54 of ribs can pass back through the slot 39 of the locking means 38, instead of being pulled further to the fourth configuration for the second vacuum. FIG. 3 further shows the cupping bell 20 with the opened end 26 with a rim. In other embodiments, such as FIG. 8, the spring means 60 can be triggered by a switch or other mechanical action.

The embodiment of the opened end 26 in FIGS. 1-8 show embodiments of a flared portion 28, 28′, 28″ made integral with the cupping bell 20. The flared portion 28 has a diameter greater than a diameter of the cylindrical body 22 of the cupping bell 20. Embodiments of opened end 26 include a generally conical flared portion. There can be an angled surface less than 90 degrees relative to the cylindrical body 22 of the cupping bell 20. The diameter of the flared portion 28 is greater, and the amount of being greater is gradual along the angled surface. The flared portion 28 engages the skin of the treatment area instead of the rim of FIG. 3. FIGS. 1, 2, 4, 6 and 8 show the flared portion 28 for the whole cylindrical body. FIG. 3 shows the flared portion above the rim. FIG. 5 shows an embodiment of the flared portion 28′ about one third of the cylindrical member 22 away from the suction chamber 30. FIG. 7 shows another embodiment of the flared portion 28″ about one fifth of the cylindrical member 22 away from the suction chamber 30. The flared portions 28, 28′, 28″ can have various proportions relative to the cylindrical body 22. The sealing engagement allows for less trauma to the skin with a more gradual increase of pressure.

The present invention includes the method for cupping with the apparatus 10. The cupping bell 20, the suction chamber 30, and the piston member 40 are assembled according to the invention. The opened end 26 of the cupping bell 20 is set against a treatment area of a patient. The treatment area is soft tissue, including skin. The blood vessels and lymphatic vessels in the treatment area are the targets of the cupping therapy. The piston member 40 actuates from the bottom end 34 toward the top end 36 with the piston member 40. Then, the locking means 38 engages with the locking notch 50. The volume of air of the cupping bell 20 is increased to create an initial vacuum with the treatment area and the cupping bell 20.

In the embodiments of the method with the apparatus of FIGS. 2 and 4-7, the step of actuating the piston member 40 includes pulling the shaft 52 and the first plurality 54 of ribs through the locking means 38. The piston member 40 is in the first configuration shown in FIG. 4. The first plurality 54 of ribs is aligned with the locking means 38 to pass through the locking means 38. The step of engaging the locking means 38 can include changing the piston member 40 from the first configuration to the second configuration. For embodiments with the locking means 38 as a slot 39 of FIGS. 4-7, the shaft 52 is rotated. The cross-section of the shaft 52 and the first plurality 54 of ribs is no longer aligned so that the piston member 40 is locked in position relative to the cupping bell 20. For other embodiments, the switch can be flipped to change from the first configuration to the second configuration of the piston member 40. The initial vacuum is the first vacuum associated with the second configuration of FIG. 5.

With embodiments of the apparatus 10 with the second plurality 56 of ribs, the method includes changing the piston member 40 from the second configuration to the third configuration, pulling the shaft 52 and the second plurality 56 of ribs through the locking means 38, and changing the piston member 40 from the third configuration to the fourth configuration. For embodiments with the locking means 38 as a slot 39 of FIGS. 4-7, the shaft 52 is rotated. The cross-section of the shaft 52 and the second plurality 56 of ribs is no longer aligned so that the piston member 40 is locked in position relative to the cupping bell 20. For other embodiments, the switch can be flipped to change from the first configuration to the second configuration of the piston member 40. The fourth configuration corresponds to another locked position of the piston member 40 relative to the cupping bell 20 and the second vacuum. The second vacuum is different from the initial vacuum and the first vacuum. In particular, the second vacuum is stronger than the first vacuum because the volume for the air in the cupping bell 20 is increased.

In the present invention, there is a continuous vacuum when the piston member 40 moves from the second configuration to the fourth configuration. There is no gap or release to preserve suction on the treatment area. The method can increase suction by adjusting the apparatus without releasing and starting over. When the cupping therapy needs to end, either in the normal course of treatment or in an emergency, the method can include releasing the initial vacuum or first vacuum by actuation of a spring means 60. The method can also include releasing the second vacuum by actuation of the spring means 60. The spring means 60 facilitates the separation of the cupping bell 20 and the treatment area.

The present invention provides an embodiment of an apparatus for cupping with a mechanically created vacuum. A piston member moves through a suction chamber in fluid connection with the cupping bell of an apparatus, such that the piston member increases the volume for the air initially in the cupping bell. Since the cupping bell is sealed to a treatment area, the treatment area is exposed to a vacuum created in the cupping bell. The amount of suction in the cupping bell is set at pre-determined levels. These predetermined levels correspond to the physical structures of the piston member. The size of the ribs on the piston member set the levels of suction so that pulling action of the piston member through the suction chamber can be mechanically limited and mechanically locked. The pulling action is made simple and standard so that users with different skill levels can achieve the same functionality of the apparatus. Additionally, levels of suction can be repeated for consistent treatments. Levels of suction can be consistently increased so that treatments can be advanced according to patient needs and preferences.

Embodiments of an apparatus for cupping with a suction chamber can also reduce trauma to the skin around the treatment area. An opened end of the cupping bell with a flared portion gradually grips the skin to create a vacuum in a cupping bell. The skin around the treatment area is less severely damaged because the change in pressure around the cupping bell is no longer as extreme. The present invention provides a safe and consistent mechanically induced vacuum for reliable and controlled cupping therapy.

The foregoing disclosure and description of the invention is illustrative and explanatory thereof. Various changes in the details of the illustrated structures, construction and method can be made without departing from the true spirit of the invention.

I claim:

1. An apparatus for cupping, comprising:
a cupping bell having a generally cylindrical body having a closed end and an opened end;
a suction chamber being in fluid connection to said closed end and having a generally tubular body having a bottom end and a top end, said suction chambering being comprised of a locking means at said top end; and
a piston member being housed in said suction chamber and movable within said suction chamber between said bottom end and said top end,
wherein said piston member is comprised of a piston skirt in sealing engagement to said suction chamber, a handle extending out of said top end of said suction chamber, and a locking notch cooperative with said locking means, said locking notch being positioned between said piston skirt and said handle.

2. The apparatus for cupping, according to claim 1,
wherein said piston member is further comprised of a shaft and a first plurality of ribs radially mounted around said shaft,
wherein said piston member has a first configuration with said locking means aligned with said locking notch so as to pass said shaft through said locking means when creating a first vacuum, and
wherein said piston member has a second configuration with said locking means engaged to said locking notch so as to hold said first plurality of ribs relative to said locking means after passing through said locking means, said first vacuum being set in said cupping bell.

3. The apparatus for cupping, according to claim 2, wherein said locking means is comprised of a slot corresponding to a cross-section of said shaft and said first plurality of ribs,
said shaft and said first plurality of ribs being aligned with said slot in said first configuration,
said first plurality of ribs being in friction fit engagement to said slot by rotation of said shaft in said second configuration.

4. The apparatus for cupping, according to claim 2, wherein said locking means is comprised of a switch and an insert,
said shaft and said first plurality of ribs being aligned with said locking means in said first configuration, said insert being in a retracted position,
said first plurality of ribs being in friction fit engagement to said locking means by actuation of said switch in said second configuration, said insert being in an extended position so as to engage said first plurality of ribs.

5. The apparatus for cupping, according to claim 2, wherein each of said ribs of said first plurality of ribs is comprised of a flange of said first plurality of ribs, each flange of said first plurality of ribs having a first length extending along said shaft.

6. The apparatus for cupping, according to claim 2,
wherein said piston member is further comprised of a second plurality of ribs radially mounted around said shaft, said second plurality of ribs being positioned between said locking notch and said piston skirt,
wherein said piston member has a third configuration with said locking means aligned with said locking notch so as to pass said shaft through said locking means when creating a second vacuum, and
wherein said piston member has a fourth configuration with said locking means engaged to said locking notch so as to hold said second plurality of ribs relative to said locking means after passing through said locking means, said second vacuum being set in said cupping bell.

7. The apparatus for cupping, according to claim 6, wherein said locking means is comprised of a slot corresponding to a cross-section of said shaft and said first plurality of ribs,
said shaft and said first plurality of ribs being aligned with said slot in said first configuration,
said first plurality of ribs being in friction fit engagement to said slot by rotation of said shaft from said first configuration to said second configuration,
said shaft and said second plurality of ribs being aligned with said slot in said third configuration by rotation of said shaft from said second configuration to said third configuration,
said second plurality of ribs being in friction fit engagement to said slot by rotation of said shaft from said third configuration to said fourth configuration.

8. The apparatus for cupping, according to claim 6, wherein said locking means is comprised of a switch and an insert,
said shaft and said first plurality of ribs being aligned with said locking means in said first configuration, said insert being in a retracted position,
said first plurality of ribs being in friction fit engagement to said locking means by actuation of said switch in said second configuration, said insert being in an extended position so as to engage said first plurality of ribs
said shaft and said second plurality of ribs being aligned with said locking means by actuation of said switch in said third configuration, said insert being in said retracted position,
said second plurality of ribs being in friction fit engagement to said locking means 38′ by actuation of said switch in said fourth configuration, said insert being in said extended position so as to engage said second plurality of ribs.

9. The apparatus for cupping, according to claim 6, wherein each of said ribs of said first plurality of ribs is comprised of a flange of said first plurality of ribs, each flange of said first plurality of ribs having a first length extending along said shaft, and
wherein each of said ribs of said second plurality of ribs is comprised of a flange of said second plurality of ribs, each flange of said second plurality of ribs having a second length extending along said shaft.

10. The apparatus for cupping, according to claim 6, wherein suction of said second vacuum is greater than suction of said first vacuum.

11. The apparatus for cupping, according to claim 9, wherein said first length of each flange of said first plurality of ribs determines said first vacuum, and wherein said second length of each flange of said second plurality of ribs determines said second vacuum.

12. The apparatus for cupping, according to claim 1, wherein said bottom end of said suction chamber extends into said cupping bell below said closed end of said cupping bell.

13. The apparatus for cupping, according to claim 1, further comprising:
a spring means between said locking means and said piston skirt.

14. The apparatus for cupping, according to claim 1, further comprising:
a flared portion on said opened end of said cupping bell, said flared portion having a diameter greater than a diameter of said cupping bell.

15. The apparatus for cupping, according to claim 14, said flared portion having an angled surface less than 90 degrees relative to said cylindrical body of said cupping bell.

16. A method for cupping, comprising the step of:
assembling a cupping apparatus 10 comprised of:
a cupping bell having a generally cylindrical body having a closed end and an opened end;
a suction chamber being in fluid connection to said closed end and having a generally tubular body having a bottom end and a top end, said suction chambering being comprised of a locking means at said top end; and
a piston member being housed in said suction chamber and movable within said suction chamber between said bottom end and said top end,
wherein said piston member is comprised of a piston skirt in sealing engagement to said suction chamber, a handle extending out of said top end of said suction chamber, and a locking notch cooperative with said locking means, said locking notch being positioned between said piston skirt and said handle.
setting said opened end of said cupping bell against a treatment area of a patient;
actuating said piston member from said bottom end toward said top end;
engaging said locking means with said locking notch so as to create an initial vacuum with said treatment area and said cupping bell.

17. The method for cupping, according to claim 16,
wherein said piston member means is comprised of a shaft and a first plurality of ribs radially mounted around said shaft,
wherein said piston member has a first configuration with said locking means aligned with said locking notch so as to pass said shaft through said locking means when creating a first vacuum, and
wherein said piston member has a second configuration with said locking means engaged to said locking notch so as to hold said first plurality of ribs relative to said locking means after passing through said locking means, said first vacuum being set in said cupping bell,
wherein said step of actuating said piston member comprises: pulling said shaft and said first plurality of ribs through said locking means, and
wherein said step of engaging said locking means comprises: changing said piston member from said first configuration to said second configuration, said first vacuum being said initial vacuum.

18. The method for cupping, according to claim 17,
wherein said piston member is further comprised of a second plurality of ribs radially mounted around said shaft, said second plurality of ribs being positioned between said locking notch and said piston skirt,
wherein said piston member has a third configuration with said locking means aligned with said locking notch so as to pass said shaft through said locking means when creating a second vacuum, and
wherein said piston member has a fourth configuration with said locking means engaged to said locking notch so as to hold said second plurality of ribs relative to said locking means after passing through said locking means, said second vacuum being set in said cupping bell, said method further comprising the steps of:
changing said piston member from said second configuration to said third configuration;
pulling said shaft and said second plurality of ribs through said locking means; and
changing said piston member from said third configuration to said fourth configuration, said second vacuum being different from said initial vacuum and said first vacuum.

19. The method for cupping, according to claim 16, wherein said cupping bell has a continuous vacuum when said piston member moves from said second configuration to said fourth configuration.

20. The method for cupping, according to claim 16, wherein said cupping apparatus further comprises a spring means between said locking means and said piston skirt, the method further comprising the step of:
releasing said initial vacuum by actuation of said spring means.
